(19) United States
(12) Patent Application Publication
(10) Pub. No.: US 2022/0387050 A1
(43) Pub. Date: Dec. 8, 2022

(54) SURGICAL MILLING CUTTER WITH IMPROVED CHIP REMOVAL

(71) Applicant: Aesculap AG, Tuttlingen (DE)

(72) Inventors: Edgar Blust, Koenigsfeld (DE); Martin Machill, Rietheim-Weilheim (DE)

(21) Appl. No.: 17/776,256

(22) PCT Filed: Nov. 12, 2020
(86) PCT No.: PCT/EP2020/081822

§ 371 Date: May 12, 2022

(30) Foreign Application Priority Data
Nov. 13, 2019 (DE) 10 2019 130 568.3

(51) Int. Cl. A61B 17/16; A61B 17/32

(52) U.S. Cl. CPC A61B 17/1615; A61B 17/32002; A61B 2017/1602

(57) ABSTRACT

A surgical milling cutter includes a shaft for rotary-driven coupling to a drive unit about a rotation axis extending in the longitudinal direction of the shaft. The surgical milling cutter has a milling cutter head arranged distally on the shaft. The milling cutter head has at least two teeth with respective cutting edges for rotary subtractive machining of tissue. The cutting edges are each designed for subtractive machining of tissue both in the distal direction and in the lateral direction. A chip space is formed as a free space between circumferentially adjacent teeth. Each chip space, on the side of the rotation axis facing towards the respective cutting edge, extends from the cutting edge into a region on the side of the rotation axis facing away from the cutting edge.

### CROSS-REFERENCE TO RELATED APPLICATIONS

This application is the United States national stage entry of International Application No. PCT/EP2020/081822, filed Nov. 12, 2020, and claims priority to German Application No. 10 2019 130 568.3, filed Nov. 13, 2019. The contents of International Application No. PCT/EP2020/081822 and German Application No. 10 2019 130 568.3 are incorporated by reference herein in their entireties.

### FIELD

The present disclosure relates to a surgical milling cutter with a shaft for rotationally driven coupling with a drive unit about a rotational axis extending in the longitudinal direction of the shaft and a cutter head arranged distally on the shaft, which has at least two teeth with cutting edges for rotationally removing tissue, said teeth each being designed for the removal of tissue both in the distal direction and in the lateral direction and extending radially outward starting at the distal part of the rotational axis of the milling cutter toward the proximal direction, wherein a chip space is formed as clearance between adjacent teeth in the circumferential direction. It also relates to a method for obtaining bone material and/or cartilage material, wherein a bone and/or cartilage is machined with a tool and the removed bone/cartilage material obtained is collected and, if required, stored and used for growing bone material.

### BACKGROUND

Surgical milling cutters are generally known from the prior art and are used for the removal of hard tissue such as bone or cartilage by driving them rotationally about a rotational axis running in their longitudinal direction. Rose-head burs or rose drills are milling tools with an approximately spherical cutter head, which is provided with many teeth carrying cutting edges, six to 14 teeth depending on the diameter. The teeth and their cutting edges converge at the distal end of the cutter head on the rotational axis of the milling cutter. As a result of the large number of teeth, their height decreases in the direction of the distal end area and is zero directly at the rotational axis. This means that there is no interdental space between the individual teeth directly at and near the distal end of the milling cutter. It may be possible to create a somewhat larger tooth height and thus somewhat larger chip spaces in the region of the distal end of the cutter head by appropriate geometric optimization. However, since the cutting edges meet in the center, i.e. at the axis of rotation of the milling head, even in the case of such optimization there are the disadvantages of difficult chip removal, relatively high heating as a result, and a severely restricted or non-existent view of the situs.

The teeth/cutting edges of such rose drills are usually produced by grinding. So-called pointed disks are used for this purpose. These usually have a grinding angle of 60° to 90° and form the interdental space through their material removal in the cutter head. The penetration depth of the pointed disk in the cutter head is set in such a way that a tooth with a corresponding cutting edge is formed from the tooth front of a previous interdental space together with the tooth back of a subsequent interdental space. The teeth are usually inserted into the cutter head with a twist, i.e. helically, so that the pointed disk does not run into the cutter shaft when grinding the proximal region of the cutter head. In this way, the teeth or the cutting edges in the proximal region of the cutter head can be formed somewhat closer to the rotational axis. The pointed disks are adjusted in such a way that the rake angle of the cutting edges is negative and is usually between −10° and −40°. The relief angle at the cutting edges is correspondingly very large and is between 40° and 70°.

A disadvantage of such rose drills in terms of production is the long running time of the grinding machine during production due to the high number of teeth/cutting edges, which leads to high production costs.

A disadvantage of this type of milling tool with regard to practical use is that due to the production-related, relatively pointed teeth and their large number, so-called ‘rattling’ may often occur when machining bones, which manifests itself by uncontrolled jumping away from the area to be machined, so that precise work is not possible or is extremely difficult.

A further disadvantage is caused by the relatively small interdental space between the teeth in the distal end region, which acts as a chip space. Bone material removed during machining accumulates in the chip space and has to be transported away from there to make room for newly removed material. If the material transport through the interdental spaces is insufficient, the chip space will become blocked and the cutting edges will be shielded by material accumulated in the chip space. As a result, further material removal is no longer possible. This is one of the reasons why it is problematic to achieve satisfactory cutting performance and cutting power in the axial direction with such milling cutter heads. This disadvantage is particularly serious since surgical procedures very often have to be performed in depth (i.e. axially) in order to remove bone in the axial direction. An obstructed cutting edge or interdental space in the center of the cutter head is difficult or impossible to detect for the surgeon, since the view is blocked by the cutter head itself. It is very difficult to clean the blocked interdental space during surgery. Once the interdental space is blocked and the surgeon then continues to work on the bone, this may lead to increased pressure forces and increased friction, which in turn leads to an increase in temperature. This is particularly critical, as temperatures above just 45° C. can already lead to coagulation of the patient's protein with known adverse consequences.

In order to reduce the latter problem, such milling cutters are flushed or cooled with liquid during operation. The liquid supports chip removal and reduces heating at the point to be machined. However, if the cutting performance is not adequate and liquid is added, increased heating can occur at specific points, which can lead to coagulation of the machined bone. In addition, excessive heating of the material located in the interdental space may occur, which may coagulate there as a result of the heating and is very difficult to remove.

A further disadvantage may be that, due to the geometry of the milling cutter, a surgeon cannot see the situs, i.e. the surgical area in the region of the tool tip. This applies to both milling cutters that are in operation and those that are at a standstill. In order to inspect the situs, it is therefore necessary to remove the milling cutter at least partially, if not completely, from the operating area.

Finally, the many deep interdental spaces of such rose drills or rose-head burs are difficult to clean in everyday clinical practice as part of their processing.

In addition to the milling cutters described above, milling tools with two cutting edges, for example so-called olive cutters or neuro cutters as well as so-called twin cutter milling tools are known in medical technology. However, these have a significantly smaller chip space.

A relatively large chip space is provided by a surgical milling cutter with only one cutting edge, a so-called single-cutter. An example of such an instrument is disclosed in U.S. Pat. No. 2,017,015 0974 A1. However, due to the provision of only one cutting edge and a design of the cutter head that is asymmetrical/eccentric to the rotational axis, its mass is distributed eccentrically so that an imbalance occurs during operation. This leads to rough, unsteady running of the milling tool, which places a high load on the handpiece and especially its bearings, and results in increased running noise.

### SUMMARY

Against this background, the object of the present disclosure is to reduce the above-mentioned disadvantages of the prior art, in particular to provide a milling cutter with improved cutting performance in the axial direction, low heat production and improved chip removal from the distal end region, in particular for high-speed applications in medicine for machining bone. A further object is to provide a milling cutter that is particularly easy to clean, in particular suitable for obtaining tissue material such as bone or cartilage for further artificial material acquisition. Finally, the disclosure is intended to provide an improved method for obtaining bone material and/or cartilage material.

This object is solved according to the present disclosure by a surgical milling cutter having a shaft for rotationally driven coupling with a drive unit about a rotational axis extending in a longitudinal direction of the shaft, in particular with a surgical handpiece, and a cutter head arranged distally on the shaft, wherein the cutter head has at least two teeth with respective cutting edges for rotationally removing tissue, wherein the cutting edges are each designed for the removal of tissue both in the axial and/or distal direction and in the radial and/or lateral direction, wherein a chip space, also referred to as interdental space, is formed as clearance in each case between teeth adjacent in the circumferential direction, wherein each chip space extends on the side of the rotational axis facing the respective cutting edge from the cutting edge into a region on the side of the rotational axis facing away from the corresponding cutting edge, at least in a distal section of the milling cutter. It can also be said that according to the disclosure, each chip space is formed on the cutting edge side of the rotational axis of the milling cutter and extends at least in a distal region on the side of the rotational axis facing away from the respective cutting edge. The surgical milling cutter according to the disclosure is preferably designed rotationally symmetrical to its rotational axis and/or with a mass distribution symmetrical to the rotational axis, so that it can be operated without imbalance.

It is a particular advantage of the disclosure that the design of the teeth or cutting edges is such that the interdental space/chip space in the distal end region of the cutter head near or at the rotational axis (at the tip) is as large as possible. This ensures good evacuation of the material removed by the milling cutter (in particular bone dust or bone chips). A liquid-based support for material evacuation can basically be reduced in most cases in an advantageous manner. Coagulation of the processed bone and also of the removed material in the interdental space as a result of excessive heating can be reliably avoided. Therefore, and due to the good accessibility of the teeth, of the interdental spaces and of the cutting edges, the milling cutter is particularly easy and good to clean. A further particular advantage of the disclosure is that the shape of the milling cutter and its chip spaces according to the disclosure enables the surgeon to have a particularly good view of the situs without having to remove all or part of the milling cutter from the operating area. Finally, the heat generation is markedly low, so that disproportionate heating, in particular to temperatures of 45° C. and more, of bone material during processing with the milling cutter according to the disclosure can be reliably avoided. An advantageous consequence of this may be that removed bone material is not damaged/does not get damaged and can therefore be used for further use, for example growing of bone material.

Advantageous embodiments of the disclosure are explained in more detail below.

One embodiment is characterized in that the cutting edges are each arcuate from distal-radial-inward to proximal-radial-outward. They can also be almost semicircular and/or extend from the widest point of the cutter head in the radial direction back to the cutter shaft in an arc. Preferably, the cutting edges of the cutter head extend radially outward starting at the distal part at the rotational axis of the milling cutter toward the proximal direction. In particular, the cutting edges of each cutter head may be formed on the edge side and may be shaped in such a way that, when the milling cutter rotates about its rotational axis (longitudinal axis), they run along an envelope curve that is preferably essentially spherical in shape. Such a milling cutter may also be referred to as a spherical cutter and is particularly well suited for uses in which material is to be removed in a lateral/radial and/or axial/distal direction. In the context of the disclosure, the cutter head may also be designed in such a way that the envelope geometry described by the cutting edges during a rotation of the milling cutter has other shapes, for example that of a flame cutter, conical cutter, cylindrical cutter or oval cutter.

According to a further embodiment, the teeth or cutting edges may each be non-helical and/or twist-free. This improves the accessibility of the interdental spaces and thus the cleaning suitability. In addition, such tooth/cutting edge shapes can be produced relatively easily, quickly, and inexpensively.

According to a further embodiment, the milling cutter may have exactly two teeth or cutting edges arranged on diametrically opposite sides of the rotational axis of the milling cutter. The cutting edges are formed on the sides of the two teeth facing away from each other, so that both cutting edges cause material removal when the milling cutter rotates about its rotational axis. The cutting side surface of the tooth is also referred to as the tooth front, while the side of the tooth facing away from the cutting edge is referred to as the tooth rear. The respective clearance on the cutting side (i.e. on the side of the tooth front) represents the chip space in the sense of the disclosure for material removed with the corresponding cutting edge. Due to the described arrangement and design of the cutting edges/teeth, the two interdental spaces are particularly large, so that particularly good transport of removed material is ensured and fast healing of non-coagulated bone can be achieved.

Such a two-toothed or two-edged design can be achieved particularly effectively and simply by machining the cutter head on both sides of the rotational axis (laterally) at least at the distal end of the milling cutter down to the rotational axis. This machining then forms a surface which is preferably flat in large parts and which forms the tooth front of the respective tooth.

Alternatively or additionally, a step or shoulder may extend starting at the distal part at the rotational axis in the proximal direction, so that the surface forming the tooth front transitions with a step or shoulder into the tooth rear of the opposite tooth. This step or shoulder forms a boundary between the tooth front of one tooth and the rear surface of the adjacent tooth in the cutting direction, i.e. the tooth rear of the other tooth. In a particularly advantageous embodiment with regard to manufacturing, the step or shoulder extends from the tip of the cutter head, as from the point of intersection of the distal end of the cutter head with the rotational axis, at an oblique angle to the rotational axis, so that the rear (proximal) region of the cutter head does not have any contact with the shaft of the milling cutter. This angle is preferably in the plane of the respective front surface, is also referred to as setting angle and is preferably in a range of 2° to 10°. In other words, the tooth front together with the circumferential relief angle forms the cutter or cutting edge. Alternatively or additionally, the tooth rear may be designed with a constant width.

The tooth front is designed and/or arranged in such a way that the rake angle at the cutting edge is 0°.

According to a further embodiment of the disclosure, the teeth may be arranged and formed transversely to the rotational axis (37) diametrically opposite each other without offset in one plane. In particular, this plane also contains the rotational axis. In particular, the teeth may be formed in such a way that a front surface of one tooth and a rear surface of the other tooth together form a flat surface. The rear surface of one tooth and the front surface of the other tooth together form a further flat surface opposite the aforementioned surface. The two surfaces may be parallel to each other. Preferably, they are parallel to each other in a direction transverse to the rotational axis and inclined to each other by a tooth thickness angle in the direction of the rotational axis.

A particularly advantageous embodiment of the disclosure with regard to material evacuation is characterized in that at least one of the cutting edges has at least one interruption or flute, in particular as a chip-breaking flute. This may be incorporated into the tooth in particular in the centripetal direction. The interruption of the cutting edges reduces their width so that penetration of the cutting edges into the bone is facilitated, which in turn leads to better cutting performance. The chip-breaking flutes of adjacent teeth/cutting edges may be designed and/or arranged offset to each other, in particular in such a way that the flutes of one tooth are located at the points corresponding to the cutting edges of the other tooth and vice versa, so that the cutting edges of the teeth are alternately involved in the removal. In this way, the size and shape of the removed chips can be determined so that the transport of removed material can be optimized. In addition, this ensures that only one cutting edge is engaged in relation to the region swept by the respective cutting edge section during its rotation about the axis of rotation, even though the milling cutter nominally has more cutting edges.

The rear side of the tooth in the cutting direction, i.e. the tooth rear, may in particular be formed and/or arranged inclined relative to the rotational axis so that the tooth widens conically from distal to proximal towards the shaft. The tooth rear may in particular be designed in the form of a flat surface, which favors accessibility, cleaning suitability, and ease of manufacture.

In particular, the tooth thickness may increase at an angle of 1° to 10° relative to the rotational axis from distal to proximal. This angle is referred to as the tooth thickness angle. This results in particularly high stability and also good cleanability. The distal thickness of the tooth or cutting edge may in particular be between 1/20 and 1/10 of the diameter of the cutter head in the radial direction. In this way, a particularly large clearance can be provided as chip space with simultaneously high stability of the tooth/cutting edge. In addition, the cutting performance and the rattling tendency can be influenced by an appropriate design of the tooth thickness. Finally, the strength of the cutter head may be defined by the tooth thickness at the required height.

A further embodiment of the disclosure is characterized in that the cutting edge has a constant relief angle in a range between 2° and 30°. Alternatively, the cutting edge may have a relief angle that varies along a course from distal to proximal, in particular in a range between 2° and 30°. The cutting edge may also have a rake angle of 0°. The circumferential relief angle forms the shape of the cutter head that defines the material removal. The cutting performance and the rattling tendency can be influenced by a corresponding design of the relief angle. By varying the relief angle over its course as described above, the cutting performance and the rattling tendency can be further positively influenced.

A particularly user-friendly embodiment is characterized in that the cutter head has a tip with an angle of 110° to 150° at its distal end, which ensures particularly good and simple centering and thus handling during use, in particular when working axially with the milling cutter tool. This angle is referred to as the tip angle. Proximally adjacent to the tip, the cutting edge/tooth may be arc-shaped, in particular circular, in the further course.

According to the disclosure, the milling cutter may be designed either as a clockwise milling cutter (right-hand cutter) or as a counterclockwise milling cutter (left-hand cutter).

With regard to a method, the solution according to the disclosure consists in a method for obtaining bone material and/or cartilage material, wherein a bone and/or cartilage is machined with a tool and the bone/cartilage material obtained is collected and, if required, stored and used for growing bone material, wherein the machining of the bone and/or cartilage is carried out with a cutting tool, in particular with a surgical milling cutter according to the disclosure, in particular according to the present description.

In summary, it can be said that the disclosure provides a surgical milling cutter, which in particular may have only two cutting edges. The interdental space is preferably designed in such a way that the removed tissue can be transported away with as little obstruction as possible. In particular, the tooth form may be designed in such a way that all material not required for the strength of the tooth is removed. In addition, surfaces of the milling cutter, in particular of the cutter head, may be designed to be smooth and flat with particular ease, which favors chip evacuation. As a result of the low temperature development and the particularly good chip evacuation, particularly gentle material removal can be achieved with the milling cutter according to the disclosure, wherein changes and/or damage to the material removed are largely, or even completely, avoided. Material removed via a milling cutter according to the disclosure is therefore particularly well suited for artificial re-growth of such material as part of the method according to the disclosure.

In particular, the disclosure brings about the following advantages:
very good cutting performance particularly in the axial direction
very good chip evacuation, since there are only two cutting edges
reduced temperature development at the cutting edge and at the tissue
very easy cleaning of the cutter head
symmetrical structure of the cutting edges
easy to clean due to very accessible surfaces
no negative rake angle
low rattling tendency
no tendency of interdental spaces to clog up
very quiet running without vibration, since the mass is arranged symmetrically to the rotational axis
due to the small number of teeth/cutting edges it can be manufactured at low cost
milling cutter that is easy and inexpensive to manufacture for high-speed applications in medicine for processing bones.

### BRIEF DESCRIPTION OF THE DRAWING FIGURES

Further features and advantages of the present disclosure result from the following exemplary and non-limiting description of the figures. These are merely schematic in nature and serve only to aid understanding of the disclosure. The following is shown:

FIG. 1 shows two perspective views of a rose drill according to the prior art,

FIG. 2 shows a perspective view of a single cutter according to the prior art,

FIG. 3 shows a perspective partial view of a distal section of a milling cutter according to a first embodiment of the disclosure,

FIG. 4 shows a side view of the milling cutter of FIG. 3,

FIG. 5 shows a cut view of the milling cutter of FIG. 3 in a direction transverse to the rotational axis,

FIG. 6 shows a top view of the milling cutter of FIG. 3,

FIG. 7 shows a front view of the milling cutter of FIG. 3,

FIG. 8 shows a partial section of the milling cutter of FIG. 3 through the rotational axis,

FIG. 9 shows a perspective view of the milling cutter of FIG. 3 from a different direction,

FIG. 10 shows a further perspective view of the milling cutter of FIG. 3 from a different direction,

FIG. 11 shows a perspective view of a different embodiment of a milling cutter according to the disclosure,

FIG. 12 shows a side view of the milling cutter of FIG. 11,

FIG. 13 shows a top view of the milling cutter of FIG. 11,

FIG. 14 shows a front view of the milling cutter of FIG. 11,

FIG. 15 shows a perspective view of a different embodiment of a milling cutter according to the disclosure,

FIG. 16 shows a perspective view of the milling cutter of FIG. 15 from a different direction,

FIG. 17 shows a perspective view of a different embodiment of a milling cutter according to the disclosure,

FIG. 18 shows an illustration corresponding to FIG. 7 with a marking of the chip spaces,

FIG. 19 shows an illustration corresponding to FIG. 14 with a marking of the chip spaces,

FIG. 20 shows a top view of a further embodiment of a milling cutter according to the disclosure,

FIG. 21 shows a cut view of the milling cutter of FIG. 20 in a direction transverse to the rotational axis,

FIG. 22 shows a cut view of the milling cutter of FIGS. 20 and 21 in a side view,

FIG. 23 shows an enlarged detail view of FIG. 22,

FIG. 24 shows a side view of a further embodiment of a milling cutter according to the disclosure,

FIG. 25 shows a top view of the milling cutter of FIG. 24,

FIG. 26 shows a front view of the milling cutter of FIGS. 24 and 25 in a direction transverse to the rotational axis, and

FIG. 27 shows an enlarged detail view of FIG. 24.

### DETAILED DESCRIPTION

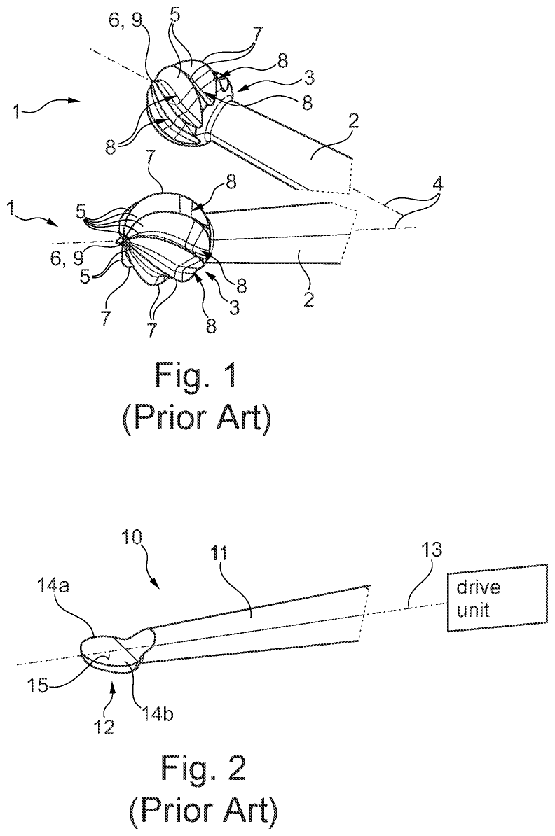

FIG. 1 shows a rose drill 1 according to the prior art in two perspective views from different directions. It has a cutter shaft 2 at the proximal side and a cutter head 3 at the distal side with an approximately spherical outer contour. The rose drill 1 has a longitudinal axis 4, which is also its rotational axis 4 during operation. In the example shown, it is provided with a total of eight teeth 5, on whose side facing away from the rotational axis 4, a cutting edge 7 is formed in each case, extending from a distal tip 6 of the cutter head 3 in the proximal direction towards the cutter shaft 2. A respective interdental space 8 is formed between adjacent teeth 5. The teeth 5 each have a twist and extend helically from distal to proximal. It can therefore be said that the teeth 5 and their cutting edges 7 converge at the distal end 9 of the cutter head at the rotational axis 4 of the rose drill 1. In the two views of FIG. 1, it is clearly shown that the height of the teeth 5 decreases towards the distal end 9 and is zero directly at the rotational axis 4 or tip 6, respectively. In other words, the depth of the interdental spaces is zero at the distal end 9 or tip 6, respectively, and increases in the proximal direction towards the cutter shaft 2. The chip space available for removed material during an axial advance in the direction of the longitudinal axis 4 of the milling cutter 1 is therefore very small near the tip 6 and not present at the tip 6, so that the interdental spaces 8 can easily become clogged with the disadvantages described at the beginning.

FIG. 2 shows a further known milling cutter 10 in the form of a so-called single cutter 10 in a perspective view. This has a cutter shaft 11 on the proximal side and a cutter head 12 on the distal side. The single cutter 10 has a longitudinal axis 13, which is also its rotational axis 13 during operation. The cutter head 12 is formed by a unilaterally inserted flattening up to the rotational axis 13 and has a cutting edge 14b, at which a substantially flat front surface 15 is formed. A recessed edge 14a is formed on the opposite side of the cutting edge 14b. This forms a clearance/free surface for the cutting edge 14b. The milling cutter is designed exclusively for clockwise rotation, i.e. it cuts only in clockwise rotation. Due to its design, the entire mass of the cutter head 11 is located on one side of the rotational axis 13, so that the mass distribution of the single cutter 10 is asymmetrical, which is associated with the disadvantages described at the beginning.

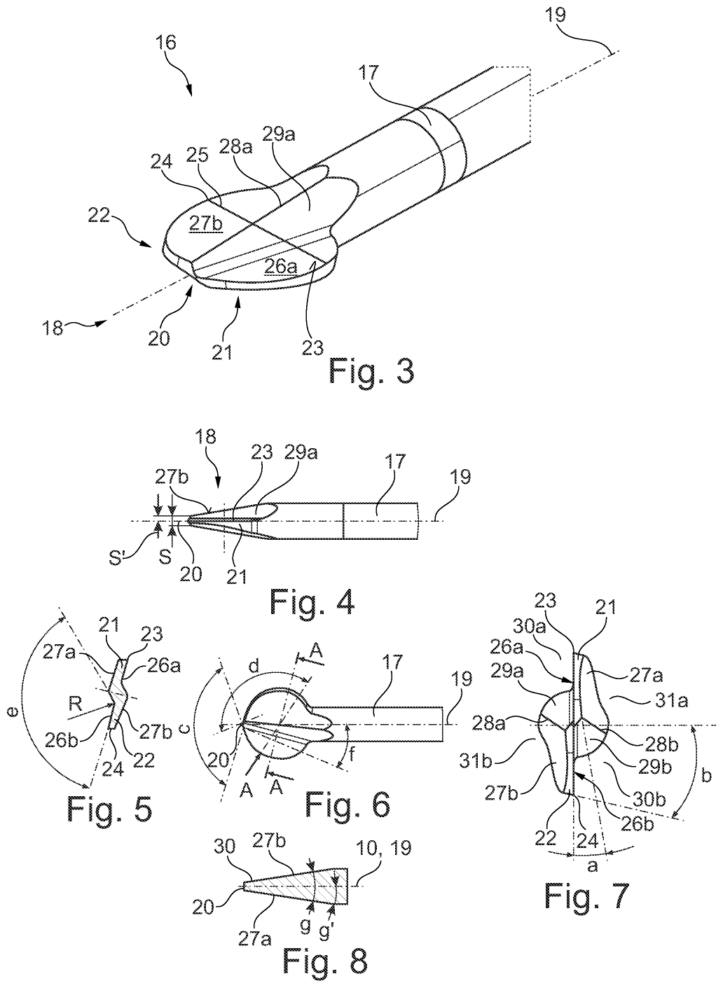

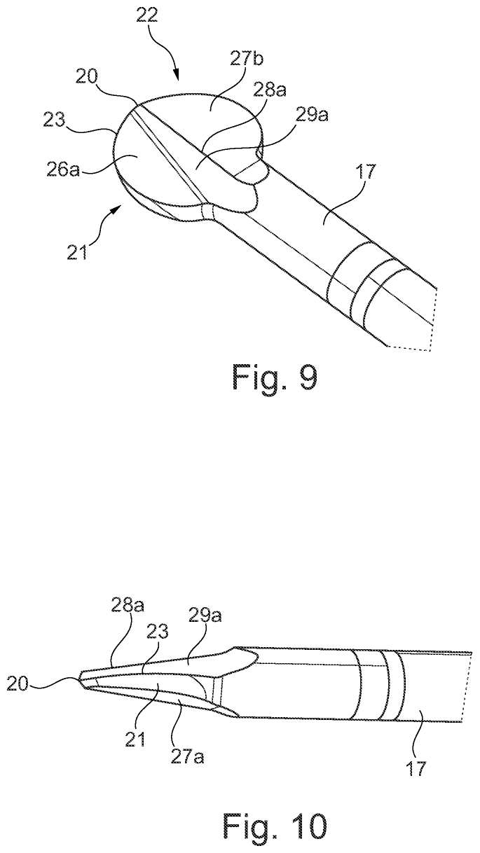

FIGS. 3, 9 and 10 show a first embodiment of a surgical milling cutter 16 according to the invention, for which views and cut views from different directions are also shown in FIGS. 4 to 8. The milling cutter 16 has a cutter shaft 17 on the proximal side and a cutter head 18 on the distal side. The milling cutter 16 has a longitudinal axis 19 which, in operation, is also its rotational axis 19. The cutter shaft 17 extends coaxially to the rotational axis 19, which centrally penetrates the cutter head 18 at its distal end 20 (see in particular FIG. 7).

The cutter head 18 has exactly two teeth 21, 22, namely a first tooth 21 and a second tooth 22. The tooth 21 has a cutting edge 23 for rotationally removing tissue. The tooth 22 has a cutting edge 24 for rotationally removing tissue. The two teeth 21, 22 and thus also the cutting edges 23, 24 are arranged on diametrically opposite sides of the rotational axis 19 of the milling cutter 16, offset from each other transverse to the rotational axis 19. The cutting edges 23, 24 are each arranged on the side of the corresponding tooth 21 or 22 facing away from the rotational axis 19 and are each arc-shaped. They begin at the distal end 20 on the rotational axis 19 and extend first from distal-radial-inward to proximal-radial-outward to then run from the widest point 25 of the cutter head 18 further in an arc towards proximal-radial-inward to the cutter shaft 17. The cutting edges 23, 24 are each non-helical and twist-free. Due to this design, the milling cutter 16 has an envelope curve of essentially spherical shape when rotating about its rotational axis 19.

The two teeth 21, 22 are identical, so that the description of the tooth 21 applies in the same way to the tooth 22. As can be seen particularly well in FIG. 7, the tooth 21 has a flat front surface 26a, which forms the frontal surface of the tooth 21 in the rotational direction, and a flat rear surface 27a, which forms the back surface of the tooth 21 in the rotational direction. The tooth 22 has a flat front surface 26b, which forms the frontal surface of the tooth 22 in the rotational direction, and a flat rear surface 27b, which forms the back surface of the tooth 22 in the rotational direction. The rotational direction is the cutting direction of the milling cutter 16. The teeth 21, 22 are offset relative to each other transversely to the rotational axis 19 (see FIG. 7) in such a way that the front surfaces 26a, 26b of the two teeth 21, 22 span a common plane in which the rotational axis 19 lies. In particular in FIG. 7 it can be clearly seen that the cutting edge 23 of the first tooth 21 and the cutting edge 24 of the second tooth 22 meet at the tip of the distal end 20 exactly where the rotational axis 19 penetrates the distal end 20. The rotational axis 19 lies in the plane of the front surface 26a and the front surface 26b.

The rear surface 27a/27b is inclined relative to the front surface 26a/26b in the axial direction by a longitudinal tooth angle g′ of between 4° and 12.5°, so that the cutter head 18 has an overall tooth thickness angle g of between 8° and 25° (see FIG. 8). In addition, the rear surface 27a/27b is inclined relative to the front surface 26a/26b in a direction transverse to the rotational axis 19 by a transverse tooth angle a between 0° and 10° (see FIG. 7). The tooth thickness S′ of the tooth 21 at the distal end 20 is diameter-dependent and amounts to about 1/10 (one tenth) of the diameter D of the cutter head 18. The tooth thickness S′ of tooth 22 at the distal end 20 is also diameter-dependent and amounts to about 1/10 (one tenth) of the diameter D of the cutter head 18. The cutter-head thickness S of the cutter head 18 thus amounts to a total of about 2/10 of the diameter D (see FIG. 4). The distal end 20 of the cutter head 18 is designed as a tip with a tip angle c of 90° to 150° (see FIG. 6). The cutting edges 23, 24 each extend from the tip at the distal end 20 over a clearance angle d of 110° to 170° in the direction of the cutter shaft 17 (see FIG. 6).

Between the front surface 26a of the tooth 21 and the rear surface 27b of the tooth 22, a step 28a or shoulder 28a with a shoulder surface 29a is formed, which extends in the proximal direction starting at the distal end 20 at the rotational axis 19. Between the front surface 26b of the tooth 22 and the rear surface 27a of the tooth 21, a step 28b or shoulder 28b with a shoulder surface 29b is formed, which extends in the proximal direction starting at the distal end 20 at the rotational axis 19. The transition from the front surface 26a, 26b to the shoulder surface 29a, 29b is rounded and is arranged at a setting angle fin the range of 2° to 10° (see FIG. 6), which is oblique relative to the rotational axis 19 and lies in the plane of the front surface 26a, 26b. The shoulder surface 29a, 29b is also arranged at a tooth front angle e in a plane transverse to the rotational axis 19 in the range of 110° to 160° (see FIG. 5). The relief angle b of the respective cutting edge 23, 24 lies in a range of 0° to 30° and is formed circumferentially, i.e. over the entire range of the clearance angle d (see FIG. 7). The transition from the front surface 26a, 26b to the shoulder surface 29a, 29b is rounded in each case, which improves material evacuation and cleaning suitability.

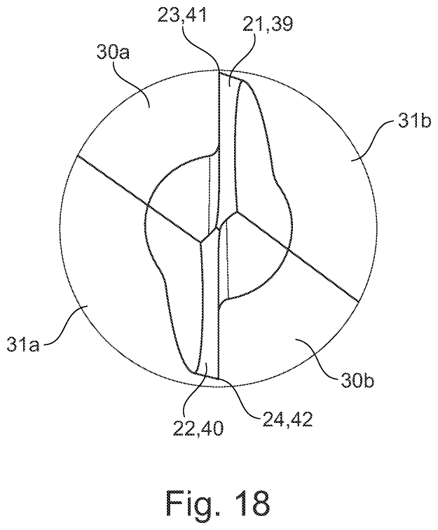

FIG. 7 clearly shows that a chip space 30a is formed as a clearance in the rotational direction in front of the first tooth 21 between its front surface 26a and its shoulder surface 29a, said chip space being available for material removed via the first tooth 21 of the milling cutter 16 and its cutting edge 23 and forming a total chip space 30a/31a together with a region 31a behind the rear surface 27b of the adjacent tooth 22. In addition, a chip space 30b is formed as a clearance in the rotational direction in front of the second tooth 22 between its front surface 26b and its shoulder surface 29b, said chip space being available for material removed via the second tooth 22 of the milling cutter 16 and its cutting edge 24 and forming a total chip space 30b/31b together with a region 31b behind the rear surface 27a of the adjacent tooth 21. FIG. 18 shows the view of FIG. 7 without reference signs, in which the clearances 30a, 30b, the regions 31a, 31b and the chip spaces 30a/31a, 30b/31b of both teeth 21, 22 of the milling cutter 16 are indicated with corresponding hatching (chip spaces 30a, 30b: hatched markings, regions 31a, 31b: checkered markings). FIG. 7 also shows that the chip space 30a, 30b on the side of the rotational axis 19 facing the respective cutting edge 23, 24 extends from the cutting edge 23, 24 to a region on the side of the rotational axis 19 facing away from the cutting edge 23, 24 and that each chip space 30a, 30b is formed on the cutting side of the rotational axis 19 and extends at least in a distal region on the side of the rotational axis 19 facing away from the respective cutting edge 23, 24. While in FIGS. 3 to 10 parts belonging to the first tooth 21 are marked by the reference sign addition a, parts belonging to the second tooth 22 are marked by the reference sign addition b, for example the front surface 26a is that of the first tooth, while the front surface 26b is that of the second tooth 22.

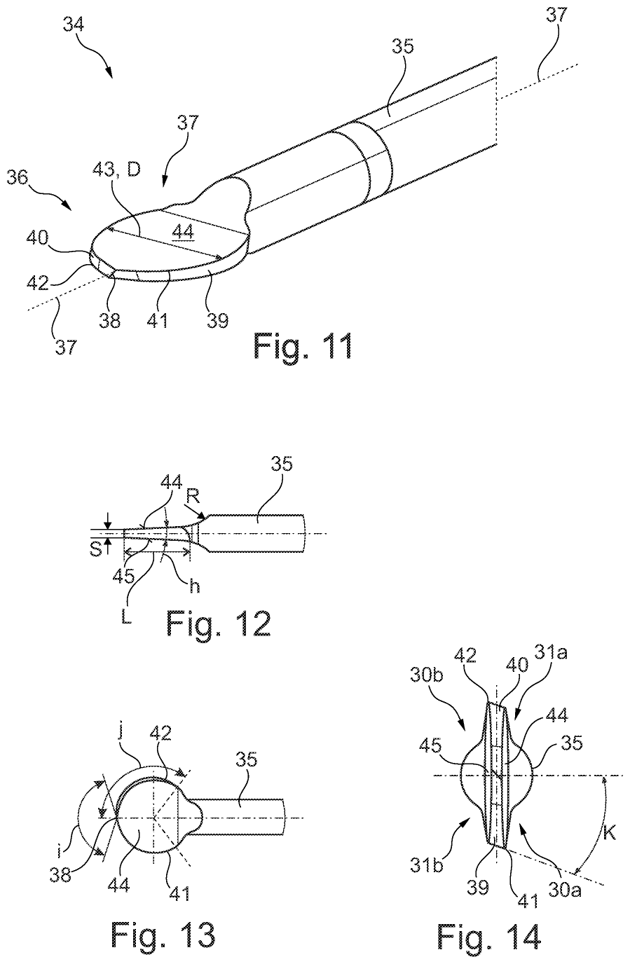

FIGS. 11 to 14 show a further embodiment of a milling cutter 34 according to the disclosure. The milling cutter 34 has a cutter shaft 35 proximally and a cutter head 36 distally. The milling cutter 34 has a longitudinal axis 37 which, in operation, is also its rotational axis 37. The cutter shaft 35 extends coaxially to the rotational axis 37, which centrally penetrates the cutter head 36 at its distal end 38 (see in particular FIG. 11).

The cutter head 36 has a first tooth 39 and a second tooth 40. The tooth 39 has a cutting edge 41 for rotationally removing tissue. The tooth 40 has a cutting edge 42 for rotationally removing tissue. The two teeth 39, 40 and thus also the cutting edges 41, 42 are arranged in a common plane, in the center of which lies the rotational axis 37, on diametrically opposite sides of the rotational axis 37 of the milling cutter 34. The cutting edges 41, 42 are each arranged on the side of the corresponding tooth 39 or 40 facing away from the rotational axis. The cutting edges 41, 42 are each arc-shaped. They begin at the distal end 38 on the rotational axis 37 and extend first from distal-radial-inward to proximal-radial-outward, then from the widest point 43 of the cutter head 34 they continue in an arc towards proximal-radial-inward to the cutter shaft 35. The cutting edges 41, 42 are each non-helical and twist-free. This shaping of the cutting edges 41, 42 gives the milling cutter 34 an envelope curve of essentially spherical shape when rotating about its rotational axis 37.

The two teeth 39, 40 are formed identically in that, as can be seen particularly well in FIGS. 11 and 12, flattenings are formed in the cutter head 36 on diametrically opposite sides of the rotational axis 37. The cutter head 36 therefore has two mutually opposite planar surfaces 44, 45, which are parallel to each other in a direction transverse to the rotational axis 37 and are inclined to each other in the direction of the rotational axis 37. The surfaces 44, 45 and the cutting edges 41, 42 are arranged and formed relative to each other in such a way that the surface 44 forms the front surface of the cutting edge 41 and the rear surface of the cutting edge 42, and the surface 45 forms the front surface of the cutting edge 42 and the rear surface of the cutting edge 41.

The surface 44 is inclined relative to the surface 45 in the axial direction by a tooth thickness angle h between 0° and 20° (see FIG. 12). The cutter head thickness S of the cutter head 34 at the tip at the distal end 38 is between 0.05 mm and 1.0 mm (see FIG. 12). The distal end 38 of the cutter head 36 is formed as a tip with a tip angle i of 90° to 160° (see FIG. 13). The cutting edges 41, 42 each extend from the tip 43 at the distal end 38 over a clearance angle j of 110° to 170° in the direction of the cutter shaft 35 (see FIG. 13). The cutter head 36 has the diameter D at the widest point. The length L of the cone, i.e. the extension of the surfaces 44, 45 in the direction of the rotational axis 37, is about 7/9D (seven-ninths of the diameter D). The surfaces 44, 45 transition into the cutter shaft 35 with a radius R, wherein the radius R corresponds to approximately half the diameter D. The relief angle k of the cutting edges 41, 42 lies in each case in a range of 0° to 30° and is formed circumferentially, i.e. over the entire region of the clearance angle j (see FIGS. 13 and 14).

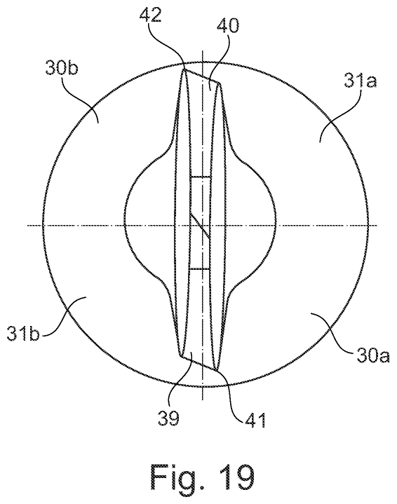

FIG. 14 clearly shows that a chip space 30a is formed as a clearance in the rotational direction in front of the first tooth 39, said chip space being available for material removed via the first tooth 39 of the milling cutter 34 and its cutting edge 41 and forming a total chip space 30a/31a together with a region 31a behind the adjacent tooth 40. In addition, a chip space 30b is formed as a clearance in the rotational direction in front of the second tooth 40, said chip space being available for material removed via the second tooth 40 of the milling cutter 34 and its cutting edge 42 and forming a total chip space 30b/31b together with a region 31b behind the adjacent tooth 39. FIG. 19 shows the view of FIG. 14 without reference signs, in which the clearances 30a, 30b, the regions 31a, 31b and chip spaces 30a/31a, 30b/31b of both teeth 39, 40 of the milling cutter 34 are marked with corresponding hatching (chip spaces 30a, 30b: hatched markings, regions 31a, 31b: checkered markings).

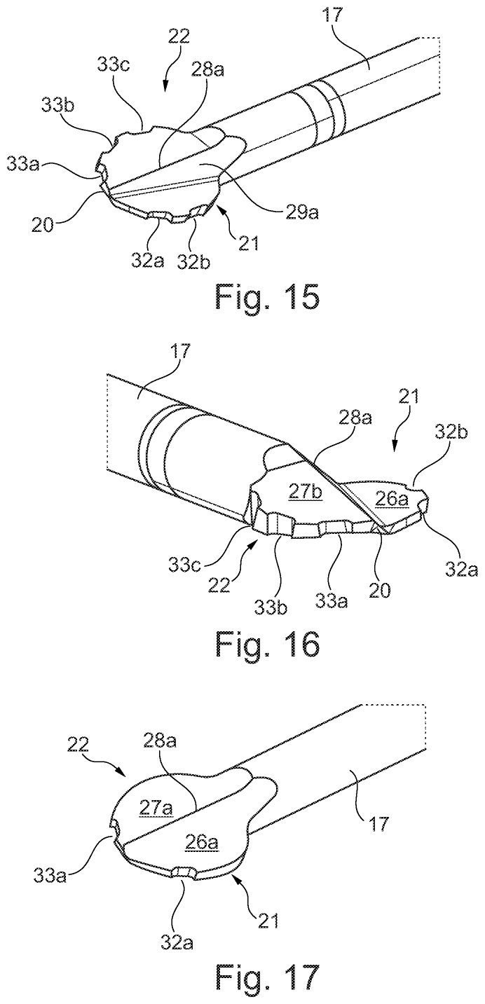

FIGS. 15 and 16 show a variant of the embodiment of FIGS. 3 to 8 described above, in which the cutting edge 23 of the tooth 21 has interruptions in the form of flutes 32a, 32b and the cutting edge 24 of tooth 22 has interruptions in the form of flutes 33a, 33b, 33c. The flutes 32a and 32b and the flutes 33a, 33b and 33c are each spaced from each other by the cutting edge parts remaining between them. They are also inserted in the centripetal direction in the corresponding tooth 21, 22 and interrupt the cutting edges 23, 24. The flutes 32a and 32b of the first tooth 21 are offset relative to the flutes 33a, 33b and 33c of the second tooth 22 in the circumferential direction of the cutting edges 23, 24 in such a way that parts at the positions of the flutes 32a, 32b of the first tooth 21 corresponding remaining cutting edge of the second tooth 22 are positioned, and at the positions of the flutes 33a, 33b, 33c of the second tooth 22 corresponding remaining cutting edge parts of the first tooth 21 are positioned. This enables a particularly uniform removal of material. With regard to the other features, the embodiments of FIGS. 3 to 8 and FIGS. 15 and 16 are similar. It is to be noted that, within the scope of the disclosure, the embodiment of FIGS. 11 to 14 may be provided with such flutes 32a, 32b 33a, 33b, 33c.

FIG. 17 shows a variant of the embodiment of FIGS. 15 and 16, in which only one flute 32a, 33a is incorporated into each of the cutting edges 23, 24. This enables particularly uniform material removal. With regard to the other features, the embodiments of FIGS. 3 to 8 and FIG. 17 are similar. It should be noted that, within the scope of the disclosure, the embodiment of FIGS. 11 to 14 may be provided with such flutes 32a, 33a.

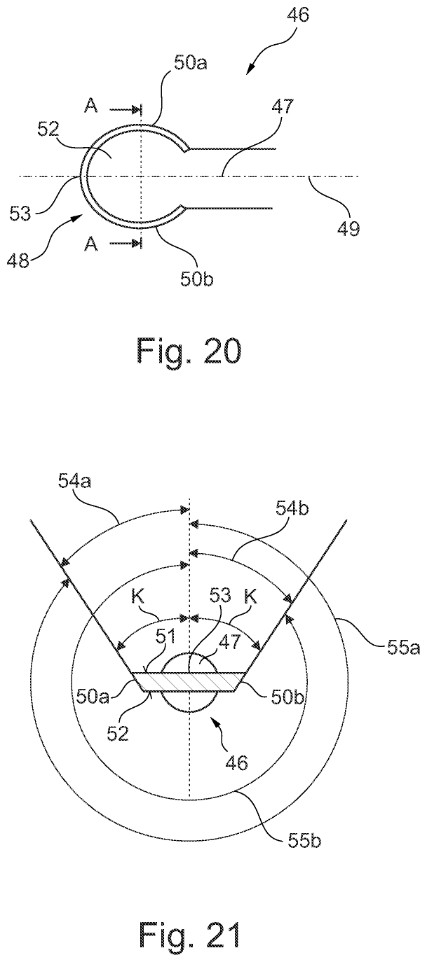

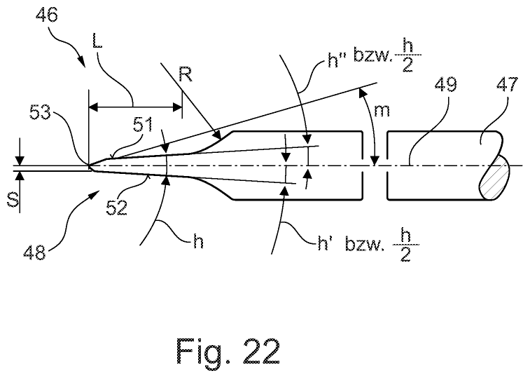

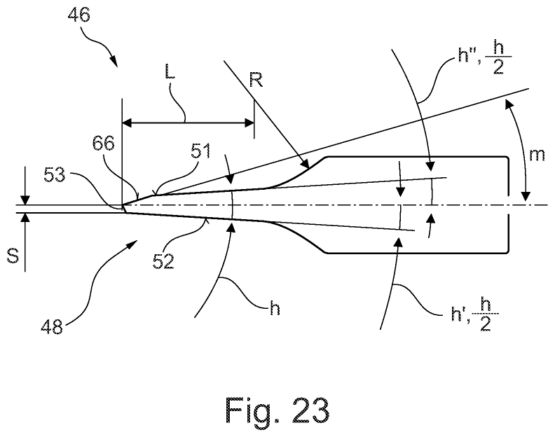

FIGS. 20, 21, 22 and 23 show a further embodiment of a milling cutter 46 according to the disclosure, which in particular is to be distinguished from the single cutter 10 shown in FIG. 2. The milling cutter 46 has a cutter shaft 47 on the proximal side and a cutter head 48 on the distal side. It also has a longitudinal axis 49 which, in operation, is also its rotational axis 49. The cutter head 48 is formed by two symmetrically opposing flattenings up to a short distance in front of the rotational axis 49 in each case, is provided with an additional pointed surface 66, which extends in the tip 53 up to the rotational axis 49 with the relief angle m, and has a cutting edge 50b, on the opposite side of which a further cutting edge 50a is formed. Both cutting edges 50a, 50b have the same shape and dimensions, in particular the same diameter, and together with the pointed surface 66 form a common front surface 51. On the side opposite the front surface, they jointly form a substantially flat rear surface 52. As shown in particular in FIGS. 21 and 22, the rear surface 52 is smaller than the front surface 51, so that a relief angle k is formed at each of the two cutting edges 50a, 50b. The front surface forms a clearance/free surface for both cutting edges 50a, 50b. The front edge 53 of the cutter head 48, which can also be referred to as tip 53, is preferably also formed as a cutting edge, so that a cutting effect can be achieved in the axial direction, i.e. in the direction of the rotational axis 49. This can be achieved, for example, by both cutting edges 50a, 50b each extending exactly as far as the rotational axis 49. The chip spaces 54a, 54b and the clearances 55a, 55b of both cutting edges 50a, 50b are shown in FIG. 21.

The milling cutter 46 is thus designed for both clockwise rotation and counterclockwise rotation, i.e. it cuts in both clockwise and counterclockwise rotation. Due to its shape, the mass of the cutter head 48 is minimized by the omission of the pointed surface 66 asymmetrical to the rotational axis 49 and results in very smooth running. In addition, the cutter head 48 may be designed with surprisingly small dimensions, in particular with a diameter smaller than, for example, 3 mm, 4 mm, or 5 mm.

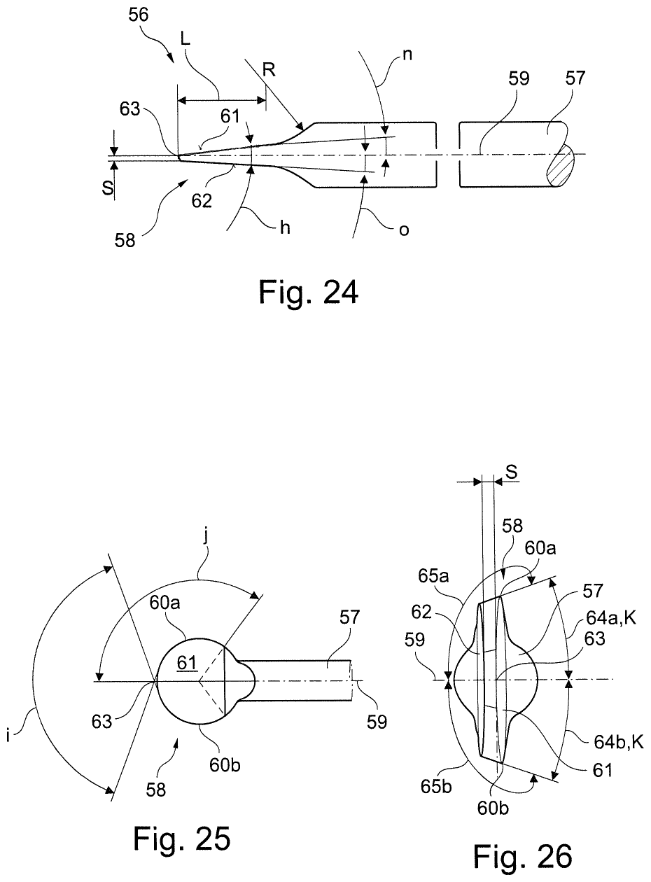

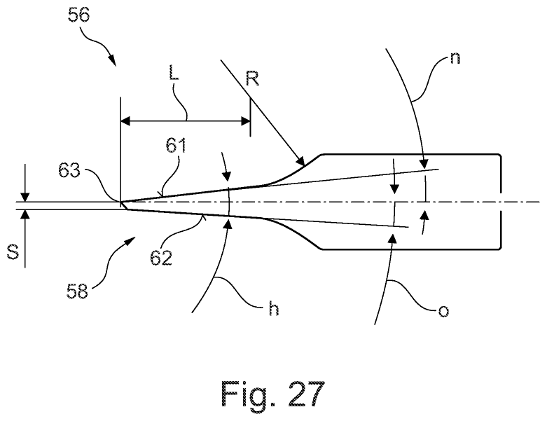

FIGS. 24, 25, 26 and 27 show a further embodiment of a milling cutter 56 according to the disclosure, which is also to be distinguished from the single cutter 10 shown in FIG. 2. The milling cutter 56 is similar to the milling cutter 46 shown in FIGS. 20 and 21 and is also designed for both clockwise rotation and counterclockwise rotation, i.e. it cuts in clockwise rotation as well as in counterclockwise rotation. It has a cutter shaft 57 on the proximal side and a cutter head 58 on the distal side. It also has a longitudinal axis 59, which in operation is also its rotational axis 59. The cutter head 58 is formed by two opposite flattenings, wherein one of the flattenings forms a front surface 61 and the other one of the flattenings forms a rear surface 62. Between the front surface 61 and the rear surface 62, a cutting edge 60b is formed on one side of the cutter head 58 and another cutting edge 60a is formed on the opposite side. The distal end of the cutter head 58 is in the form of a tip 63. The special feature of the milling cutter 56 is that its front surface 61 lies on the rotational axis 59 at the tip 63. It can be said that the front surface 61, the cutting edges 60a, 60b, and the rotational axis 59 meet at tip 63. The two cutting edges 60a, 60b have the same shape and dimensions, in particular the same diameters. As shown in particular in FIG. 24, the rear surface 62 is smaller than the front surface 61, so that a relief angle m is formed at each of the two cutting edges 60a, 60b. The front surface forms a respective clearance/free surface for both cutting edges 60a, 60b. The front edge or tip 63 of the cutter head 58 is preferably also formed as a cutting edge, so that a cutting action can be effected in the axial direction, i.e. in the direction of the rotational axis 59. The chip spaces 64a, 64b and the clearances 65a, 65b of both cutting edges 60a, 60b are drawn in FIG. 24.

The cutter head 58 has a cutter-head thickness S at its tip 63. Starting from the tip 63, the front surface 61 and the rear surface 62 are inclined relative to each other by a tooth thickness angle h. In addition, the front surface 61 is inclined relative to the rotational axis 59 by an upper tooth thickness angle n and the rear surface 62 is inclined relative to the rotational axis 59 by a lower tooth thickness angle o, so that the relationship h=n+o applies.

Due to its shape, the milling cutter 56 has a somewhat less favorable mass distribution with respect to eccentricity than the milling cutter 46 shown in FIGS. 20 and 21, but it has better cutting edge properties in the direction of the rotational axis 59. Also, the cutter head 58 may be formed with surprisingly small dimensions, in particular with a diameter smaller than, for example, 3 mm, 4 mm, or 5 mm.

1.-16. (canceled)

17. A surgical milling cutter comprising:
a shaft coupled to a drive unit about a rotational axis, the rotational axis extending in a longitudinal direction of the shaft; and
a cutter head arranged distally on the shaft,
the cutter head comprising at least two teeth, each of the at least two teeth comprising a cutting edge for rotationally removing tissue,
the cutting edges each configured to remove tissue both in a distal direction and in a lateral direction,
the at least two teeth separated from one another by a chip space that forms a clearance in a circumferential direction,
the at least two teeth or cutting edges being non-helical and twist-free, and
the chip space extending on a side of the rotational axis facing one of the cutting edges from the cutting edge into a region on a side of the rotational axis facing away from said one of the cutting edges.

18. The surgical milling cutter according to claim 17, wherein the cutting edges are each arcuate from distal-radial-inward to proximal-radial-outward.

19. The surgical milling cutter according to claim 17, wherein the at least two teeth consist of two teeth arranged on diametrically opposite sides of the rotational axis.

20. The surgical milling cutter according to claim 19, wherein the two teeth are offset relative to each other transversely to the rotational axis.

21. The surgical milling cutter according to claim 20, wherein the two teeth are offset relative to each other transversely to the rotational axis in such a way that front surfaces of the two teeth span a common plane in which the rotational axis lies.

22. The surgical milling cutter according to claim 21, wherein each of the front surfaces is substantially flat and/or comprises a step extending in a proximal direction starting at a distal part at the rotational axis and is arranged relative to the rotational axis at a setting angle of 2° to 10° lying in the common plane.

23. The surgical milling cutter according to claim 17, wherein the at least two teeth lie transversely to the rotational axis diametrically opposite each other without offset.

24. The surgical milling cutter according to claim 17, wherein the cutting edge of at least one of the at least two teeth comprises at least one interruption or flute that is incorporated into said at least one of the at least two teeth in a centripetal direction and causes a reduction in length of the cutting edges and thus serves to reduce a feed force.

25. The surgical milling cutter according to claim 17, wherein a thickness of each of the at least two teeth increases with a tooth thickness angle of 1° to 10° relative to the rotational axis from distal to proximal.

26. The surgical milling cutter according to claim 17, wherein a distal thickness of each of the at least two teeth is between 1/20 and 1/10 of the diameter of the cutter head in a radial direction.

27. The surgical milling cutter according to claim 17, wherein each cutting edge has a constant relief angle of 2° to 30° along the cutting edge or the cutting edge has a relief angle in a range of 2° to 30° that varies along a course of the cutting edge.

28. The surgical milling cutter according to claim 17, wherein each cutting edge has a rake angle of 0°.

29. The surgical milling cutter according to claim 17, wherein the cutter head has a tip with a tip angle of 110° to 150° at its distal end.

30. The surgical milling cutter according to claim 17, wherein the surgical milling cutter is rotationally symmetrical to the rotational axis.

31. The surgical milling cutter according to claim 17, wherein the cutting edges comprise a first cutting edge which effects tissue removal in a first direction of rotation and a second cutting edge which effects tissue removal in a second direction of rotation opposite to the first direction of rotation.

32. The surgical milling cutter according to claim 17, wherein the cutter head has a flame-shaped, cone-shaped, olive-shaped or roller-shaped cross-sectional shape in a cross-section through the rotational axis.

33. A method for obtaining bone material and/or cartilage material using the surgical milling cutter according to claim 17.

34. A surgical milling cutter comprising:
a shaft coupled to a drive unit about a rotational axis, the rotational axis extending in a longitudinal direction of the shaft; and
a cutter head arranged distally on the shaft,
the cutter head comprising at least two teeth, each of the at least two teeth comprising a cutting edge for rotationally removing tissue,
the cutting edges each configured to remove tissue both in a distal direction and in a lateral direction,
the at least two teeth separated from one another by a chip space that forms a clearance in a circumferential direction, and
the at least two teeth being offset relative to each other transversely to the rotational axis in such a way that front surfaces of the at least two teeth span a common plane in which the rotational axis lies.
